(19) United States
(12) Patent Application Publication
(10) Pub. No.: US 2002/0197227 A1
(43) Pub. Date: Dec. 26, 2002

(54) Anti-frizz hair care compositions

(76) Inventor: William F. Scholz, Scottsdale, AZ (US)

Correspondence Address: William F. Scholz, 10135 E. Desert Cove Avenue, Scottsdale, AZ, 85260, US

(21) Appl. No.: 10/152,416

(22) Filed: May 21, 2002

Related U.S. Application Data
(60) Provisional application No. 60/293,118, filed on May 23, 2001.

(51) Int. Cl. A61K 7/06; A61K 7/11

(52) U.S. Cl. 424/70.12

(57) ABSTRACT

Hair care compositions are described that provide frizz control and maintain volume for naturally curly hair in high humidity environments. The compositions comprise from about 4% to 17% of a high viscosity flexible polydimethylsiloxane polymer and a mixture of volatile and non-volatile carrier fluids.

### CROSS-REFERENCE TO RELATED APPLICATIONS

This patent was originally filed as a Provisional application. The application No. is 60/293,118, filed on May 23, 2001.

### STATEMENT REGARDING FED SPONSORED R&D

The present invention was not federally sponsored.

### TECHNICAL FIELD

The present invention relates to hair care compositions that control hair frizz and changes in hair volume with changing humidity conditions due to the inclusion of specific medium and high molecular weight polydimethylsiloxane polymers, and mixtures of volatile and non-volatile silicone-based carrier fluids.

### BACKGROUND OF THE INVENTION

Curly hair tends to become frizzy and significantly expand in volume when exposed to high humidity conditions or changes in the ambient humidity level. These changes in shape and body of the hair are believed to result from increased water uptake into the hair follicle. For temporary control, styling mousse, gels, lotions, and spray compositions have been employed to combat these conditions. Unfortunately, these products usually leave the hair stiff and unmanageable after application and negatively affect ease of combing and hair feel. To improve hairstyle retention properties without the adverse effects described above, more recent hair care compositions have utilized silicone-based constituents. These have been described in many publications including U.S. Pat. No. 4,673,568, Grollier, issued Jun. 16, 1987, U.S. Pat. No. 4,894,224, Kuwata, issued Jan. 16, 1990, U.S. Pat. No. 4,902,499, Bolish, issued Feb. 20, 1990, U.S. Pat. No. 4,906,459, Cobb, issued Mar. 6, 1990, U.S. Pat. No. 4,983,383, Maksimoski, issued Jan. 8, 1991, U.S. Pat. No. 5,997,853, Bolich, issued Dec. 7, 1997, and U.S. Pat. No. 4,764,363, Bolich, issued Aug. 16, 1998.

It has now been discovered that hair care compositions comprising a mixture of flexible medium and high molecular weight silicone polymers in volatile and non-volatile carrier fluids control hair frizz and volume in both high humidity environments and where there are rapid changes in the ambient humidity level. These compositions can be applied to either wet or dry hair, and provide enhanced control until they are washed from the hair with shampoo.

The object of the present invention is to provide hair care compositions that reduce hair frizz and control hair volume without leaving hair stiff or with a tacky feel. This is accomplished by providing hair care compositions using specific mixtures of hydrophobic silicone-based polymers and carrier fluids that effectively envelop the hair strand and prevent the transpiration of water either into or out of the hair follicle. These and other objects will become readily apparent from the detailed description which follows.

Unless otherwise indicated, all percentages herein are by weight.

### SUMMARY OF THE INVENTION

The present invention relates to hair care compositions comprising from about 4% to 17% of a high molecular weight flexible silicone polymer, from about 10% to 25% of a non-volatile medium molecular weight silicone-based carrier fluid, and from about 58% to 86% of a volatile low molecular weight silicone-based carrier fluid and mixtures thereof.

### DESCRIPTION OF THE INVENTION

The compositions described in this application temporarily alter the state of the hair by limiting moisture movement into and out of the hair follicle. This application describes hair care compositions comprising mixtures of flexible silicone-based gum polymers (polydimethylsiloxane polymers) and volatile and non-volatile silicone-based carrier fluids that reduce hair frizzing and control hair volume, especially in high humidity environments.

High humidity environments cause curly hair to frizz uncontrollably and expand in volume. Because many curly-haired individuals desire a more controlled style, the hair-care industry has focused on this issue for many years. However, to date there has been no practical commercial solution available. This invention disclosure provides a solution for control of hair under these environmental conditions. These compositions prevent or substantially reduce the rate at which water either moves into or out of the hair follicle with changing ambient humidity levels. Reduced moisture transpiration provides the anti-frizz and volume-control properties apparent when using this product. Hydrophobic coatings are effective means to reduce moisture transpiration. Silicone fluids and gums are very hydrophobic and have been used previously in the hair care industry. The key attribute in the present application is the use of highly flexible silicone-based gums that do not make hair stiff and unmanageable but rather provide a very thin moisture barrier while retaining the desired manageability properties.

These products can be applied to either wet or dry hair. They can be applied as a mousse, or from a pump or spray applicator. The optimum benefit is realized when the product is applied to the hair while wet and reapplied after drying. For extended control, the product can be reapplied to dry hair after the initial application.

The volatile carrier fluids are diluents to the silicone gum components and transiently provide a conditioning effect to the hair, which improves wet combing prior to evaporation. The non-volatile carrier fluids provide a longer term conditioning effect, and help maintain a long lasting, uniform, and very thin coverage of the gum components on each individual strand of hair by reducing the inherent viscosity of the gum.

The flexible gum component(s) are non-volatile and provide the water barrier properties of the formulation. Additional benefits to these products include improved hair softness and shine. These additional beneficial properties result primarily from the gum components. The essential, as well as optional, components are described below.

### Silicone Gum Mixtures

The use of a single silicone gum is efficacious in this application, but the preferred formulations incorporate at least two different molecular weight (size) silicone gums. The most preferred formulations incorporate a mixture of two trimethyl silyl end-blocked (dimethicone) gum polymers. The all-dimethicone blends provide enhanced hair frizz control properties over the dimethyl silanol end-blocked (dimethiconol) polymers used singularly or in blends with the dimethicone silicone gums. The preferred product uses a combination of Dow Corning Silicone Gum Blends. The specific gum blends used in the preferred formulation are 1411 Fluid and 1428 Fluid. However, blends of Dow Coming 1401, 1404, and similarly comprised polymers are also effective as anti-frizz materials.

Dow Corning 1411 Fluid is a clear blend comprised of 15% trimethyl silyl terminated (dimethicone) high viscosity gum and 85% cyclopentasiloxane (D5) solvent. Dow Coming 1428 Fluid is also a clear blend comprised of 33% high viscosity dimethicone gum and an intermediate viscosity non-volatile dimethicone fluid (see description below). The preferred formulations incorporate between 0% and 35% of the 1411 Fluid and 15 and 35% of the 1428 Fluid. The most preferred formulations incorporate about 30% 1411 Fluid and about 28% 1428 Fluid.

In a practical sense, the key element for frizz control is uniform coverage of the flexible gum components on each strand of hair. Providing a range of molecular weight polymers by using at least two flexible siloxane polymers in the product formulation improves coverage, in part, by lowering the overall inherent viscosity of the active ingredients in the product. The lower molecular weight polymers have a lower intrinsic viscosity, and improve the coverage of the larger molecules on the outer surface of the hair follicle. The range of polymer sizes and specific chemistries used in the preferred formulations provide unique hair control properties. Trimethyl silyl terminated polymers are preferred in these formulations. The use of a flexible polymer improves overall coverage, which further reduces water transpiration across the hair follicle. An added benefit to using a flexible polymer is that the hair remains in control in high humidity environments but is not stiff. Most other commercial products that claim an anti-frizz benefit make the hair stiff and unworkable after drying on the hair.

Another factor that differentiates these formulations from the prior art is the total percentage of high molecular weight silicone gum(s) that is incorporated into the formulation. These formulations balance the viscosity of the applied solution with the delivery of an adequate total amount of active ingredient to provide the desired effect.

### Silicone Carrier Fluids

The compositions of the present invention also comprise volatile and non-volatile silicone-based carrier fluids used either individually or in mixtures. The total amount of carrier fluid used in these formulations is between about 80% and 95%. The carrier fluid can be added into the formulation as either an individual component or in combination with the silicone gums described above. The term “volatile” means that the material has a measurable vapor pressure, and will evaporate from the formulation after the product is applied to the outer surface of the hair. The term “non-volatile” means that the carrier fluid will not substantially evaporate after the product is applied to the hair. The carrier fluids can be either cyclic or linear polydimethylsiloxanes. The volatile components are generally cyclic siloxane molecules with viscosities generally less than about 10 cP at 25° C. The preferred formulation incorporates Dow Corning 245 Fluid. This product is primarily a cyclopentasiloxane (D5) silicone fluid. However, cyclotetrasiloxane, cyclohexasiloxane, and other similarly designed products are also functional. The formulation composition can incorporate between about 30% and about 85% 245 Fluid. The most preferred formulations incorporate about 42% 245 Fluid.

The non-volatile carrier fluid is an intermediate viscosity, linear dimethicone-based material that helps the product spread when applied. This material has a similar chemical structure to the dimethicone gums described above. The structural similarity is important since it improves the dissolution of the high molecular weight gum into the carrier fluid. However, because the carrier is also non-volatile, it also contributes to the overall functionality and long-term performance of the product. The non-volatile carrier fluid can be introduced into the formulation either as an independent component or in combination with one or both of the silicone gums. The formulation compositions can incorporate between about 10% and 25% non-volatile carrier fluid with the most preferred formulations incorporating about 19%.

The most preferred compositions were determined empirically by practical testing in commercial hair care salons.

I claim:

1. A hair care composition comprising:
(a) from about 4% to about 17% of a high viscosity silicone polymer, and
(b) from about 10% to about 25% of non-volatile silicone-based carrier fluid, and
(c) from about 58% to about 86% of a volatile silicone-based carrier fluid.

2. A hair care composition according to claim 1 wherein the high viscosity silicone polymer is selected from a group consisting of organic substituted siloxane gums and siloxane elastomers.

3. A hair care composition according to claim 2 wherein the high viscosity silicone polymer is a trimethyl silyl terminated polydimethylsiloxane gum.

4. A hair care composition according to claim 1 wherein the non-volatile silicone-based carrier fluid is an intermediate viscosity linear dimethicone fluid.

5. A hair care composition according to claim 1 wherein the volatile silicone-based carrier fluid is a cyclic silicone containing from about 3 to about 7 silicone atoms.

6. A hair care composition comprising:
(a) from about 10% to about 15% of a high viscosity silicone polymer, and
(b) from about 10% to about 20% of non-volatile silicone-based carrier fluid, and
(c) from about 65% to about 80% of a volatile silicone-based carrier fluid.

7. A hair care composition comprising:
(a) about 14% of a high viscosity silicone polymer, and
(b) about 19% of non-volatile silicone-based carrier fluid, and
(c) about 67% of a volatile silicone-based carrier fluid.

8. A hair care composition according to claim 1 in the form of a spray including their use in pump sprays.
